(19) United States
(12) Patent Application Publication
(10) Pub. No.: US 2026/0014364 A1
(43) Pub. Date: Jan. 15, 2026

(54) INTEGRATED NEEDLE FREE CONNECTOR SECUREMENT DEVICE FOR EASY MANEUVERABILITY

(71) Applicant: CareFusion 303, Inc., San Diego, CA (US)

(72) Inventors: Praveen NALAWADE, Bengaluru (IN); Shishir Prasad, Ramsey, NJ (US); Jason Andrew Wine, Brea, CA (US); Manish Kumar, Bengaluru (IN)

(21) Appl. No.: 18/770,418

(22) Filed: Jul. 11, 2024

(51) Int. Cl. A61M 39/10

(52) U.S. Cl. CPC A61M 39/1011

(57) ABSTRACT

Fluid delivery securement systems, needleless connector securement systems, and methods for regulating fluid delivery with these systems are disclosed. The fluid delivery securement system comprises a catheter securement system and a needleless connector securement system. The catheter securement system and the needleless connector securement system are primary and secondary securement devices that prevent harmful movement of the fluid delivery devices. The catheter securement system includes a catheter securement device adhered to the patient and a catheter hub comprising one or more catheters. The needleless connector securement system comprises a securement device adhered to the patient. The securement device is movably coupled to a bracket, which can be coupled to different needleless connectors. The needleless connectors, in turn, are coupled to catheters. The bracket can rotate and translate the needleless connector and the catheter relative to the securement device, which facilitates clinician access to the needleless connector and the catheter.

The present disclosure relates generally to needleless fluid connector systems and, more particularly, to securement devices for needleless fluid connector systems.

### BACKGROUND

Medical treatments often include the infusion of a medical fluid (e.g., a saline solution or a liquid medication) to patients using an intravenous (IV) catheter that is connected through an arrangement of flexible tubing and fittings, commonly referred to as an “IV set,” to a source of fluid (e.g., an IV bag). Alternatively, medical fluid can be delivered to patients using syringes.

Both IV sets and syringes can couple to patients through needleless fluid connectors attached to catheters. When a needleless fluid connector and a catheter are inserted into a patient, a clinician often has to secure the needleless fluid connector and the catheter to the patient (i.e., when the medical fluid is being delivered over an extended period of time) to prevent displacement.

### SUMMARY

Aspects of the present disclosure provide a medical fluid delivery securement system that includes a catheter securement system and a needleless connector securement system, which secure the catheter and the needleless connector to the patient to prevent displacement and dislodgement of the medical fluid delivery devices and, thus, injury to the patient. This is because when a medical fluid delivery device (e.g., a catheter or a needleless connector) is secured to a patient (e.g., adhered to the patient’s arm), the forces required to retain the medical fluid delivery device are greater than the forces required to remove it.

In accordance with at least some embodiments disclosed herein is the realization that medical fluid delivery devices are often at risk of being inadvertently removed from the patient due to forces applied to different components of the medical fluid delivery system (e.g., when the IV, the tubing, or the catheter get caught on the patient’s clothing, the patient’s bedding, or other equipment). For this reason, it is important to provide primary and secondary securement of the medical fluid delivery devices to prevent displacement or dislodgement of the medical fluid delivery devices as well as injury and infection to the patient. The primary securement device directly holds the catheter in place on the skin near the insertion site to prevent movement or displacement of the catheter near the insertion site. Equally important and absent from traditional securement systems is secondary securement, which holds the tubing and extension lines in place. This is because, by holding the tubing and the extension lines connected to the catheter in place with the secondary securement device, forces applied to the medical fluid delivery devices are absorbed by the secondary securement device, which limits the amount of force applied to the primary securement device. In turn, this limits the likelihood of displacement or dislodgement of the catheter at the insertion site as well as the likelihood of injury and infection to the patient. In the present disclosure, the catheter securement system is the primary securement system, and the needleless connector securement system is the secondary securement system.

Also in accordance with at least some embodiments disclosed herein is the realization that existing securement devices do not include a means for managing the catheter during saline-administer-saline (SAS) or saline-administer-saline-heparin (SASH) workflow. With existing securement devices, clinicians must hold the catheter after it has been disinfected in order to prevent contamination of the catheter. This is extraordinarily inconvenient because during this time, clinicians also have to handle other medical devices in order to execute the SAS or SASH workflow. In the present disclosure, the needleless connector securement system is designed to hold the disinfected catheter in place so that the clinician can execute the SAS or SASH workflow with both hands without having to worry about contamination of the catheter.

Accordingly, aspects of the present disclosure provide a fluid delivery securement system, the system comprising: a needleless connector comprising a port at a proximal end of the needleless connector; a bracket comprising an inner surface configured to couple to the proximal end of the needleless connector, an outer surface, and one or more engagement features on the outer surface; a securement device comprising a first side, a second side, and one or more wings protruding from the first side, wherein the one or more wings comprise one or more reciprocal engagement features for receiving the one or more engagement features on the bracket, wherein the one or more engagement features are movably coupled to the one or more reciprocal engagement features such that the bracket and the needleless connector can move relative to the securement device; an adhesive pad engageable with the second side of the securement device, wherein the adhesive pad comprises an engagement side configured to engage the adhesive pad with the second side of the securement device and an adhesive side configured to adhere the securement device to a patient; a catheter securement device comprising a different adhesive pad configured to adhere the catheter securement device to the patient; and a catheter hub coupled to the catheter securement device and comprising one or more catheters, wherein one catheter of the one or more catheters is configured to be removably coupled to the needleless connector at the port.

Aspects of the present disclosure provide a needleless connector securement system, the system comprising: a bracket comprising one or more engagement features and configured to receive a needleless connector; a securement device comprising a first side, a second side, and one or more wings protruding from the first side, wherein the one or more wings comprise one or more reciprocal engagement features for receiving the one or more engagement features on the bracket, wherein the one or more engagement features are movably coupled to the one or more reciprocal engagement features such that the bracket can move relative to the securement device; and an adhesive pad engaged with the second side of the securement device.

Some instances of the present disclosure provide a method for regulating fluid delivery, the method comprising: providing a needleless connector, the needleless connector comprising a port at a proximal end of the needleless connector and an opening at a distal end of the needleless connector; providing a bracket coupled to the proximal end of the needleless connector, the bracket comprising an inner surface, an outer surface, and one or more engagement features on the outer surface; providing a securement device coupled to the inner surface of the bracket, the securement device comprising a first side, a second side, one or more wings protruding from the first side, and one or more reciprocal engagement features on each of the one or more wings; providing an adhesive pad coupled to the second side of the securement device; receiving, at the port of the needleless connector, a catheter; adhering, with the adhesive pad, the securement device, the bracket, and the needleless connector to a patient; and rotating and/or translating the bracket and the needleless connector relative to the securement device to access the distal end of the needleless connector and couple a fluid delivery device to the opening.

Accordingly, the present application addresses several operational challenges encountered in prior medical fluid delivery devices and systems that involve dangling extension lines and tubing. Securing the medical fluid delivery devices in place at both primary and secondary locations prevents complications related to inadvertent pulling on the dangling extension lines and tubing. Further, the present application addresses operational challenges related to managing the medical fluid delivery devices during SAS and SASH workflow in order to prevent contamination of the disinfected medical fluid delivery devices.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set and a syringe coupled to a patient, in accordance with the aspects of the present disclosure.

FIGS. 2A and 2B illustrate a perspective view and an exploded perspective view of the fluid delivery securement system, in accordance with some aspects of the present disclosure.

FIG. 3 illustrates an exploded view of the needleless connector securement system, in accordance with some aspects of the present disclosure.

FIGS. 4A and 4B illustrate side views of the different rotational positions of the needleless connector securement system and the catheter, in accordance with some aspects of the present disclosure.

FIGS. 5A and 5B illustrate the engagement features and the reciprocal engagement features when the needleless connector securement system and the catheter are in different rotational positions, in accordance with some aspects of the present disclosure.

FIGS. 6A and 6B illustrate perspective views of the adhesive pad with gripping ridges, in accordance with some aspects of the present disclosure.

FIGS. 7A, 7B, and 7C illustrate perspective and top views of the adhesive pad with guiding ridges and elastomeric ribs, in accordance with some aspects of the present disclosure.

FIGS. 8A and 8B illustrate perspective views of the securement device with a key and the adhesive pad with a slot, in accordance with some aspects of the present disclosure.

FIG. 9 illustrates an embodiment of the fluid delivery securement system that includes an extension line aligner, in accordance with some aspects of the present disclosure.

FIG. 10 illustrates a flowchart showing a method for regulating fluid delivery, in accordance with some aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Both primary and secondary securement of medical fluid delivery devices are essential to in delivering medical fluid to a patient via a catheter. Medical fluid delivery devices inevitably require tubing and extension lines, which can get caught on other medical equipment or inadvertently pulled by patients. When the tubing and extension lines get caught or pulled on, forces are applied to the medical fluid delivery system at the insertion site, which can move, or even completely remove, the catheter from the insertion site. This movement can cause abrasions and infections to the patient at the insertion site. To prevent this movement, both primary and secondary securement systems are crucial to secure the placement of the medical fluid delivery devices. Primary securement systems secure the placement of the catheter (or a catheter hub connecting multiple catheters to the patient) at the insertion site in order to minimize displacement of the catheter. Secondary securement systems are also crucial, even though they are not utilized in conventional securement systems. Secondary securement systems secure the medical fluid delivery devices (e.g., extension lines, needleless connectors, etc.) to the patient at positions further from the insertion site. Secondary securement systems reduce the impact at the insertion site caused by forces applied to the medical fluid delivery devices by absorbing part or all of the magnitude of those forces at a location further from the insertion site.

Another essential aspect of medical fluid delivery is managing the catheter (or catheters) during the SAS and SASH workflow. Traditional medical fluid delivery systems require clinicians to hold disinfected medical fluid delivery devices (e.g., catheters) in their hands (to prevent contamination of the catheters) while executing the rest of the SAS or SASH workflow. This means that clinicians must execute the rest of the SAS or SASH workflow with only one hand. It is inconvenient (and, in some situations, dangerous) for the clinician to execute the SAS or SASH workflow with only one hand. Additionally, catheters are more likely to get contaminated if clinicians have to hold the catheters while completing other tasks. For this reason, it is valuable for a medical fluid delivery securement system to hold the medical fluid delivery device for the clinician. It is even more valuable if the medical fluid delivery securement system holds the medical fluid delivery device in a position in which the clinician can disinfect the medical fluid delivery device.

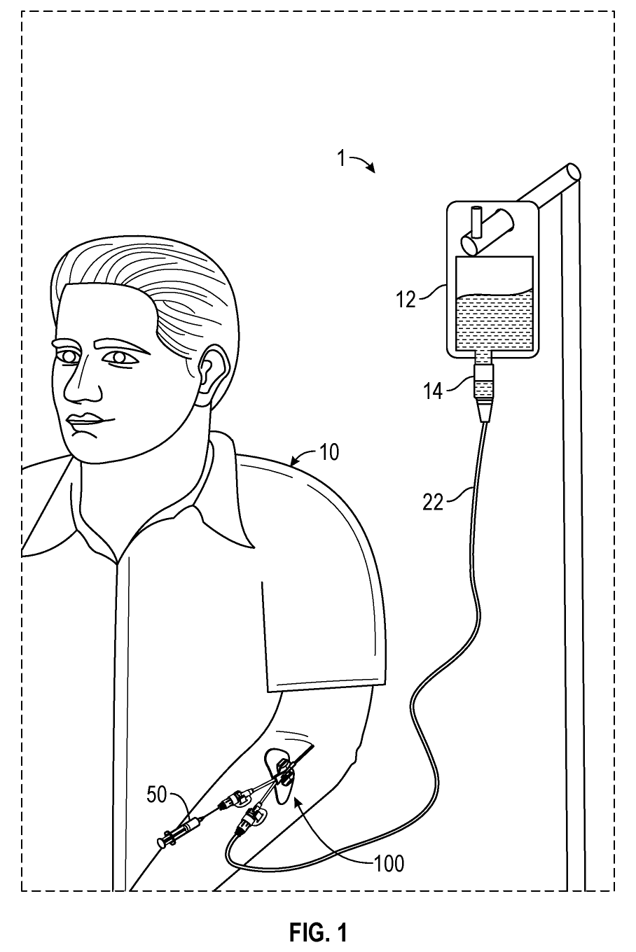

Referring now to the figures, FIG. 1 illustrates an IV set and a syringe coupled to a patient 10, in accordance with the aspects of the present disclosure. The IV set 1 includes a medical fluid bag 12, a drip chamber 14, and tubing 22. The tubing 22 extends between the drip chamber 14 and a fluid delivery securement system 100 of the IV set 1. The fluid delivery securement system 100 resists unintended dislodgement or disconnection of the tubing 22, the catheter 102 (which can also be referred to as a catheter hub), and the needleless connector 104 from the patient 10.

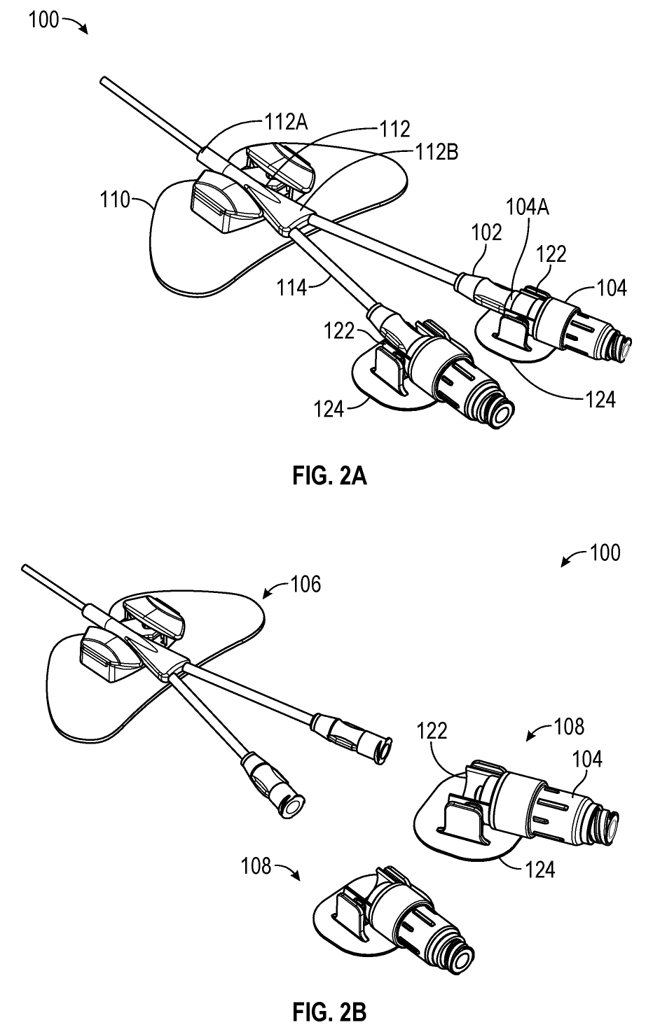

FIGS. 2A and 2B illustrate a perspective view and an exploded perspective view of the fluid delivery securement system, in accordance with some aspects of the present disclosure. As shown in FIG. 2B, the fluid delivery securement system 100 is made up of two systems: the catheter securement system 106 and the needleless connector securement system 108. The catheter securement system 106 is a primary securement that directly holds the catheter in place on the skin in order to stabilize the position of the catheter. The needleless connector securement system 108 is a secondary securement. In other words, the needleless connector securement system 108 is an additional anchor for the tubing 22 or the extension lines 114. Ordinarily, in a medical fluid delivery system without secondary securement, when a force is applied to the tubing 22 or the extension lines 114 (e.g., inadvertent tugging or pulling), a force is also applied to the primary securement, which can apply force to the insertion site. This can cause discomfort or injury to the patient. If the force is strong enough, it can even dislodge the medical fluid delivery system, which can cause abrasions to the insertion site, infect the insertion site, and contaminate the medical fluid delivery system—all of which can lead to failure of the IV. The secondary securement absorbs some of the force applied to the tubing 22 or the extension lines 114, which reduces the force applied to the primary securement and reduces the likelihood of the aforementioned complications.

The catheter securement system 106 may include a catheter securement device 110 which adheres to the patient 10 and holds the catheter hub 112. The catheter hub 112 includes the catheter 102. Utilizing a catheter hub 112 allows attachment and removal of various extensions, reduces the total surface area of the extravascular portion of the catheters and the predominant site of a contaminant’s origin (as compared to connecting catheters at multiple insertion sites), facilitates improved placement of insertion-site dressing, and substantially reduces potential for snagging, irritation, etc. by reducing the number of dangling catheter extensions required to connect multiple catheters to the patient).

The catheter securement device 110 may have a flat base with a bottom side and a top side. The bottom side may include an adhesive substance or otherwise be configured to adhere to the patient 10. The flat base of the catheter securement device 110 may be wide in order to provide a lot of surface area contact between the catheter securement device 110 and the skin of the patient 10, which secures the catheter securement system 106 in place relative to the patient 10. The top side of the flat base is configured to receive the catheter hub 112. The catheter hub 112 can be snapped, slid, threadingly fastened, or otherwise secured into the top side of the flat base of the catheter securement device 110. The proximal end 112A of the catheter hub 112 is coupled to a line that is inserted into the arm of the patient 10, and the distal end 112B of the catheter hub 112 may be connected to one or more catheters 102 by one or more extension lines 114. In FIGS. 2A and 2B, the catheter hub 112 is connected to two catheters 102 by two extension lines 114.

The catheter securement system 106 is attachable to the needleless connector securement system 108 by the catheter 102. Each catheter 102 is removably couplable to a needleless connector 104, as shown by the catheters 102 being coupled to the needleless connectors 104 in FIG. 2A and disconnected in FIG. 2B. The catheter 102 is coupled to the needleless connector 104 at a port located at the proximal end 104A of the needleless connector 104. The needleless connector 104 is coupled to a bracket 122, a needleless connector securement device 124 (also referred to as a securement device 124), and an adhesive pad 120.

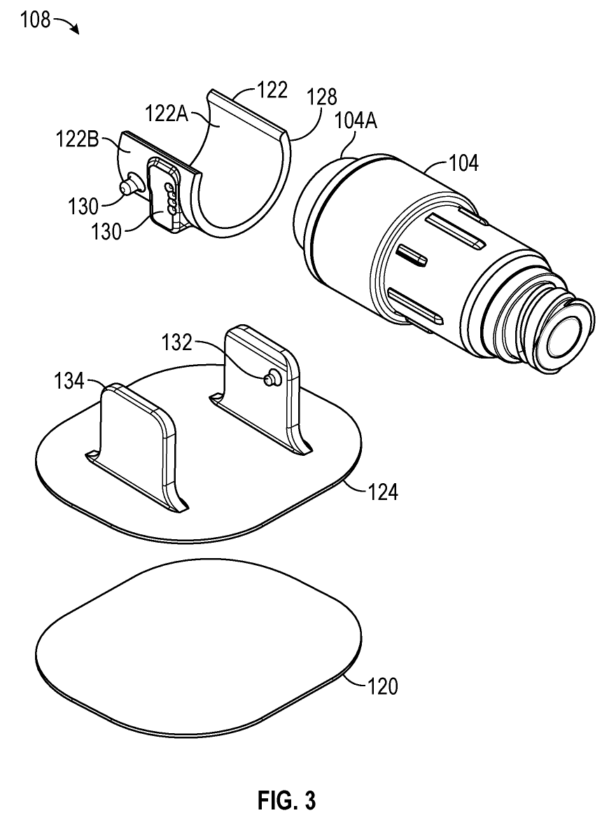

FIG. 3 illustrates an exploded view of the needleless connector securement system, in accordance with some aspects of the present disclosure. The needleless connector securement system 108 may include the needleless connector 104, a bracket 122, a securement device 124, and an adhesive pad 120. The needleless connector 104 is snapped, slid, threadingly fastened, or otherwise secured to an inner surface 122A of the bracket 122 at the proximal end 104A of the needleless connector 104 such that the bracket 122 cradles the needleless connector 104. In some embodiments, the needleless connector 104 is interference fitted to the bracket 122 (i.e., the inner surface 122A of the bracket 122 is compressed around the proximal end 104A of the needleless connector 104A). Because the needleless connector 104 is coupled to the bracket 122 by an interference fit, multiple types and shapes of needleless connectors 104 can be connected to and locked into the bracket 122.

The bracket 122 may include one or more projecting flanges 128 that secure the needleless connector 104 to the bracket 122. The projecting flanges 128 snap to an external surface of the needleless connector 104 and hold the bracket 122 against the needleless connector 104. The projecting flanges 128 are extensions at the open portion of the bracket 122. The projecting flanges 128 compress the needleless connector 104 to hold the needleless connector 104 in place and are compatible with multiple types, shapes, and sizes of needleless connectors 104.

The bracket 122 may have a generally cylindrical shape. The cross-section of the bracket 122 may be an open circle with a hollow center, as shown in FIG. 3. The cross-section of the bracket 122 may also be a closed circle with a hollow center (e.g., a ring or a hoop). The bracket 122 also has an outer surface 122B and one or more engagement features 130 positioned on the outer surface 122B.

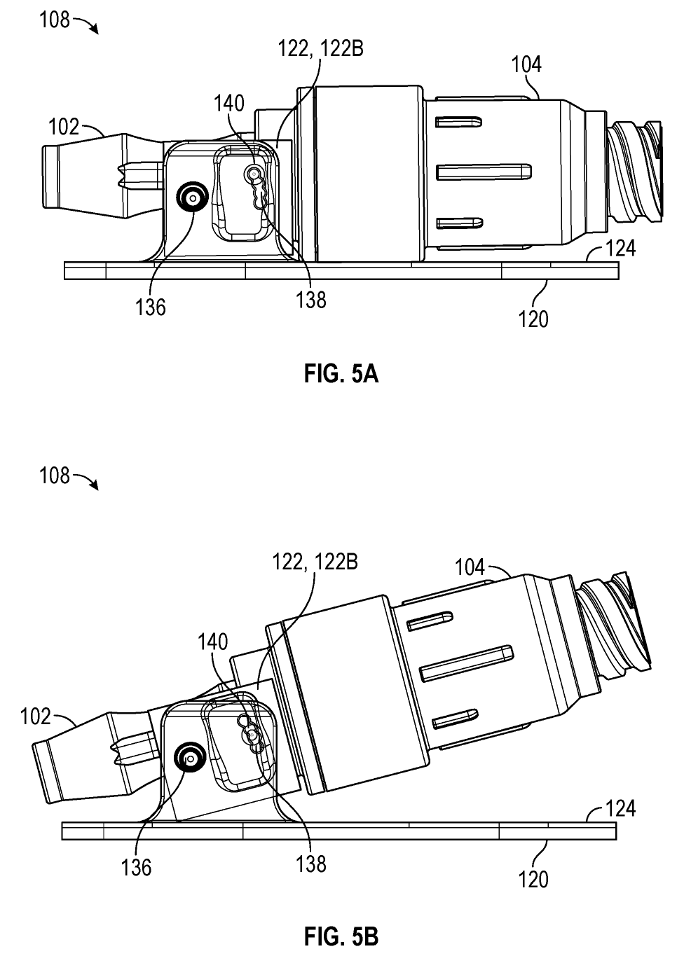

The bracket 122 is movably coupled to the securement device 124. The engagement features 130 on the outer surface 122B of the bracket 122 are movably coupled to one or more reciprocal engagement features 132 on the securement device 124. The securement device 124 includes a flat base with a first side and a second side. The flat base of the securement device 124 may be wide in order to provide a lot of surface area contact between the securement device 124 and the skin of the patient 10, which secures the needleless connector securement system 108 in place relative to the patient 10. The first side of the securement device 124 includes one or more wings 134 protruding from the first side. The wings 134 are panels, prongs, consoles, or boards that extend perpendicularly from the flat base. In some embodiments, there are two parallel wings 134. The wings 134 include reciprocal engagement features 132 that are movably coupled to the engagement features 130 on the outer surface 122B of the bracket 122. In some embodiments, each wing 134 has one or more reciprocal engagement features 132. In some embodiments, only some of the wings 134 have one or more reciprocal engagement features 132. The engagement features 130 and the reciprocal engagement features 132 are described in further detail below with respect to FIGS. 5A and 5B.

The second side of the securement device 124 is configured to adhere to the patient 10. By adhering the securement device 124 (and, thus, the needleless connector securement system 108) to the patient 10, the extension line 114 can be held tautly against the arm of the patient 10 so that the catheter 102 and the needleless connector 104 are not dangling off the arm of the patient 10. This configuration reduces the likelihood of the extension line 114, the catheter 102, and the needleless connector 104 getting tangled (e.g., in clothing or other medical equipment), pulled on, or broken, which, in turn, reduces the likelihood of catheter dislodgement, infiltration, phlebitis, migration and abrasion to the skin of the patient 10.

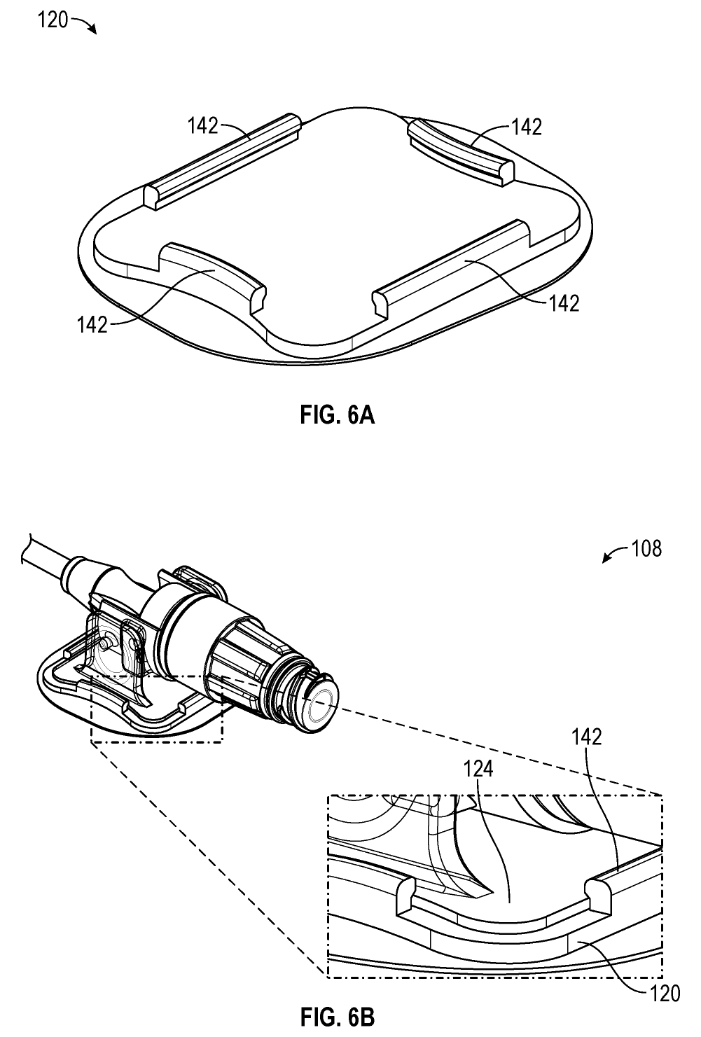

In some embodiments, an adhesive pad 120 is applied to the second side of the securement device 124. The adhesive pad 120 may have a generally square shape, but other shapes are also possible. The adhesive pad 120 may be described as having two sides: an engagement side that engages with the second side of the securement device 124 and an adhesive side configured to adhere the needleless connector securement system 108 to the patient 10. In some embodiments, the adhesive pad 120 may be sticky on both the engagement side and the adhesive side such that the adhesive side of the adhesive pad 120 sticks to the second side of the securement device 124, and a protective film or layer may be peeled off the engagement side to expose the adhesive surface and adhere the needleless connector securement system 108 to the patient 10. In other embodiments, the second side is coated in an adhesive material. In other embodiments, the adhesive pad 120 is a reusable pad or a reusable silicone pad. The reusable pad and the reusable silicone pad may be peeled off the second side of the securement device. With a reusable adhesive pad, the user may also change the position of the needleless connector securement system 108 relative to the catheter securement system 106 while the fluid delivery securement system 100 is in use. For example, the user can change the position of the needleless connector securement system 108 from near the catheter securement system 106 to a position near the wrist, which is useful when a user is self-injecting in a homecare setting. The needleless connector system 108 can also be moved back to the original position near the catheter securement system 106 after the self-injection. Additional embodiments of the adhesive pad 120 are discussed below with reference to FIGS. 6A-8B.

The user may also apply extra adhesive pads 120 to the arm of the patient 10 so that the user may stow the needleless connector securement system 108 when the needleless connector securement system 108 is not in use. For example, the user may apply adhesive pads 120 near the insertion site and near the wrist of the patient 10. When the needleless securement system 108 is in use (e.g., when the user needs to access the needleless connector 104), the user may place the needleless securement system 108 near the wrist of the patient 10. When the user does not need to access the needleless connector 104, the user may remove the securement device 124 (as well as the bracket 122 and the needleless connector 104) from the adhesive pad 120 near the wrist and secure it to the adhesive pad 120 near the insertion site. For this use case, the extension line 114 may be a long or flexible extension line 114. Long and flexible extension lines 114 accommodate a wide variety of needleless connector 104 placements and reduce the peeling loads applied when the extension line 114 is accidentally pulled.

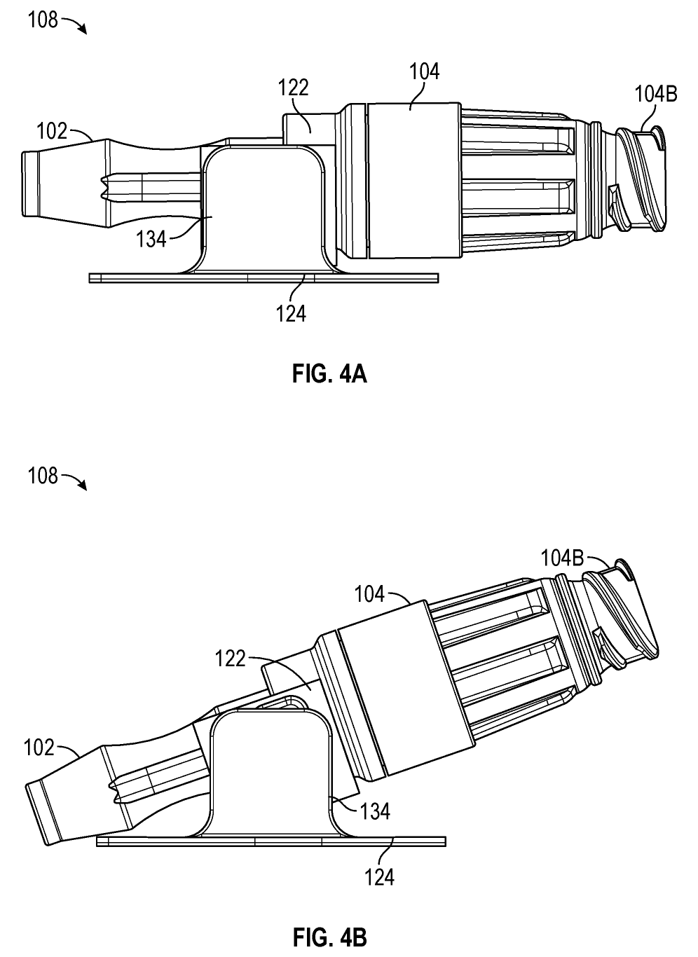

FIGS. 4A and 4B illustrate side views of the different rotational positions of the needleless connector securement system and the catheter, in accordance with some aspects of the present disclosure. In FIG. 4A, the catheter 102 and the needleless connector 104 are horizontal (i.e., parallel to the flat base of the securement device 124 and the arm of the patient 10). This position can be considered the resting or default position of the fluid delivery securement system 100. In this default, horizontal position, fluid will flow from the IV set 1 into the catheter 102 and through the needleless connector 104.

In FIG. 4B, the catheter 102 and the needleless connector 104 are rotated counterclockwise. In this position, the distal end 104B of the needleless connector is a sufficient height from the arm of the patient 10 to allow a clinician to manually disinfect the needleless connector 104 and connect a fluid delivery device (e.g., a syringe 50 or tubing 22 as shown in FIG. 1) to the needleless connector 104.

FIGS. 5A and 5B illustrate the engagement features and the reciprocal engagement features when the needleless connector securement system and the catheter are in different rotational positions, in accordance with some aspects of the present disclosure. The one or more engagement features 130 are positioned on the outer surface 122B of the bracket 122. The one or more engagement features 130 are designed to couple with one or more reciprocal engagement features 132 on the wings 134 of the securement device 124. The engagement features 130 are movably coupled to the reciprocal engagement features 132 such that the bracket 122 and the needleless connector 104 can move relative to the securement device 124 and the arm of the patient 10. The bracket 122 and the needleless connector 104 can rotate and/or translate relative to the securement device 124 and the arm of the patient 10.

The engagement features 130 and the reciprocal engagement features 132 may include one or more intrusions, protrusions, keys, and/or slots located on the outer surface 122B of the bracket 122 and on the wings 134 of the securement device 124. These features can be seen in FIG. 3. The one or more engagement features 130 may include an intrusion that receives a protrusion, where the protrusion is the one or more reciprocal engagement features 132. Alternatively, the engagement feature 130 may include a protrusion, while the reciprocal engagement feature 132 includes an intrusion. In some embodiments, there are multiple intrusions and protrusions between the bracket 122 and the wings 134. In other embodiments, the bracket 122 and the wings 134 may each have both intrusions and protrusions. In this instance, each intrusion and protrusion on the bracket 122 (i.e., the engagement features 130) are configured to movably couple with each protrusion and intrusion on the wings 134 (i.e., the reciprocal engagement features 132).

As shown in FIGS. 5A and 5B, the engagement features 130 and reciprocal engagement features 132 may form a pivot 136 that facilitate rotation. The bracket 122 and the needleless connector 104 may rotate relative to the securement device 124 via the pivot 136. Specifically, the needleless connector 104 may be rotated clockwise and counterclockwise around the pivot 136. This rotation of the needleless connector 104 provides space between the opening at the distal end 104B of the needleless connector 104 and the arm of the patient 10 so that the user can access the opening of the needleless connector. When the catheter 102 is coupled to the needleless connector 104 (as shown in FIGS. 5A and 5B), the catheter 102 may be rotated similarly. The rotation of the catheter 102 provides a similar space between the proximal end of the catheter 102 and the arm of the patient 10 so that the user can access the catheter 102.

Similarly, the one or more engagement features 130 may include a key that fits into a slot, where the slot is the one or more reciprocal engagement features 132. Alternatively, the engagement feature 130 may include a slot, while the reciprocal engagement feature 132 includes a key. In some embodiments, there are multiple keys and slots between the bracket 122 and the wings 134. In other embodiments, the bracket 122 and the wings 134 may each have both keys and slots. In this instance, each key and slot on the bracket 122 (i.e., the engagement features 130) are configured to movably couple with each slot and key on the wings 134 (i.e., the reciprocal engagement features 132).

In some embodiments, the slots are straight, vertical slots that facilitate translation. When the key travels along the straight, vertical slot, the bracket 122 and the needleless connector 104 move vertically relative to the arm of the patient 10. The needleless connector 104 can be pulled away from the arm of the patient 10 (i.e., forcing the key to travel up the length of the vertical slot) to provide a user with access to the needleless connector 104. When the user no longer needs to access the needleless connector 104, the user can push the needleless connector 104 toward the arm of the patient 10 (i.e., forcing the key to travel down the length of the vertical slot). In some embodiments, the vertical slot includes notches. The notches catch the key to lock the key at different heights along the vertical slot so that the needleless connector 104 can rest at various heights relative to the arm of the patient 10. The notches may also serve to provide tactile feedback to the user, which helps the user identify how far the key is traveling up and down the vertical slot.

In some embodiments, the slots are straight, horizontal slots that facilitate translation. When the key travels along the straight, horizontal slot, the bracket 122 and the needleless connector 104 move horizontally relative to the arm of the patient 10. In some embodiments, the horizontal slot includes notches. The notches catch the key in different positions along the length of the horizontal slot so that the needleless connector 104 can rest at various positions relative to the arm of the patient 10. The notches may also serve to provide tactile feedback to the user, which helps the user identify how far the key is traveling along the length of the horizontal slot. As shown in FIGS. 5A and 5B, the one or more engagement features 130 may include a curved slot 138 with notches while the one or more reciprocal engagement features 132 may include a key 140. This configuration of the engagement features 130 and the reciprocal engagement features 132 may facilitate rotation, translation, or both rotation and translation. The key 140 fits inside the curved slot 138 and travels along the curved slot 138 to rotate and translate the bracket 122 and the needleless connector 104 relative to the securement device 124. The curvature of the curved slot 138 enables the rotational movement, while the length of the curved slot 138 enables the translational movement. As the key 140 travels along the curved slot 138, the key 140 contacts the notches of the curved slot 138. In this way, the notches provide a user of the fluid delivery securement system 100 (e.g., a clinician or a patient 10 self-administering in a homecare setting) with tactile feedback as the key 140 moves along the curved slot 138, which informs the user of the extent of the movement of bracket 122 and the needleless connector 104 relative to the securement device 124 and the patient 10. The notches also serve to lock the bracket 122 and the needleless connector 104 in various positions relative to the securement device 124 and the patient 10. In some embodiments, the one or more engagement features 130 includes the key 140, while the one or more reciprocal engagement features 132 includes the curved slot 138.

FIGS. 6A and 6B illustrate perspective views of the adhesive pad with gripping ridges, in accordance with some aspects of the present disclosure. The gripping ridges 142 are designed to fit around the perimeter of the flat base of the securement device 124. Specifically, the securement device 124 can be snapped on the adhesive pad 120 and held in place by the gripping ridges 142, as shown in FIG. 6B. The gripping ridges 142 are located along the perimeter of the engagement side of the adhesive pad 120. In some embodiments, the gripping ridges 142 are continuous along the perimeter of the engagement side of the adhesive pad. In other embodiments, the adhesive pad 120 has multiple, separate gripping ridges 142. As shown in FIG. 6A, the adhesive pad 120 has a generally square shape and includes a gripping ridge 142 on each side of engagement side of the adhesive pad 120. Each gripping ridge 142 protrudes vertically from the engagement side of the adhesive pad 120. The top of each gripping ridge 142 includes an overhang or lip that extends toward the center of the adhesive pad 120. As shown in FIG. 6B, the securement device 124 can be snapped into the adhesive pad 120 such that the overhangs of the gripping ridges 142 rest on top of the flat base of the securement device 124 to hold the securement device 124 in place relative to the adhesive pad 120.

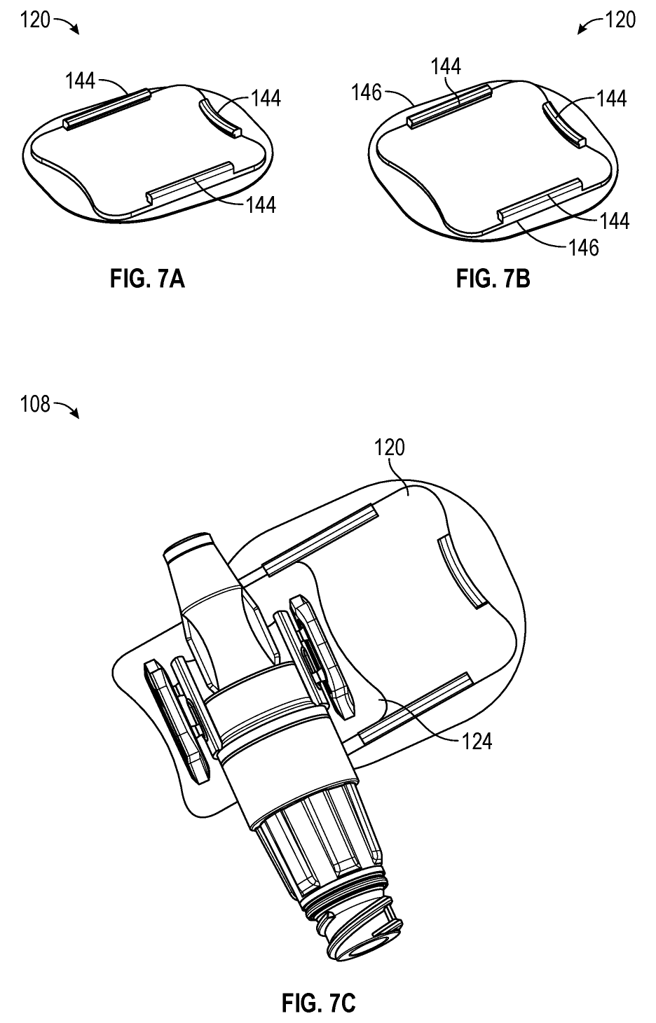

FIGS. 7A, 7B, and 7C illustrate perspective and top views of the adhesive pad with guiding ridges and elastomeric ribs, in accordance with some aspects of the present disclosure. The guiding ridges 144 are designed to receive the perimeter of the flat base of the securement device 124. In particular, the securement device 124 can be slid onto the adhesive pad via an opening in the guiding ridges 144 and held in place by the guiding ridges 144, as shown in FIG. 7C. The guiding ridges 144 are located along the perimeter of the engagement side of the adhesive pad 120. The guiding ridges 144 may include a single, continuous guiding ridge 144 or multiple, separate guiding ridges, as shown in FIGS. 7A and 7B. The guiding ridges 144 include an opening at which the adhesive pad 120 can receive the securement device 124. Each guiding ridge 144 protrudes vertically from the engagement side of the adhesive pad 120. In some embodiments, the top of each guiding ridge 144 includes an overhang that extends toward the center of the adhesive pad 120. As shown in FIG. 7C, the securement device 124 can be slid into the adhesive pad 120 such that the overhangs of the guiding ridges 144 rest on top of the flat base of the securement device 124 to hold the securement device 124 in place relative to the adhesive pad 120.

In some embodiments, one or more of the guiding ridges may also include an elastomeric rib 146. The elastomeric rib 146 is an elastomeric panel that extends from the top of a guiding ridge 144. When the securement device 124 is slid into the adhesive pad 120, the securement device 124 applies an outward force to the guiding ridges 144 (i.e., the securement device 124 pushes out on the guiding ridges 144 and applies a force having a direction opposite the center of the adhesive pad 120). The stiffness of the elastomeric ribs 146 opposes this force by pushing against the securement device 124 to maintain the position of the securement device 124 relative to the adhesive pad 120.

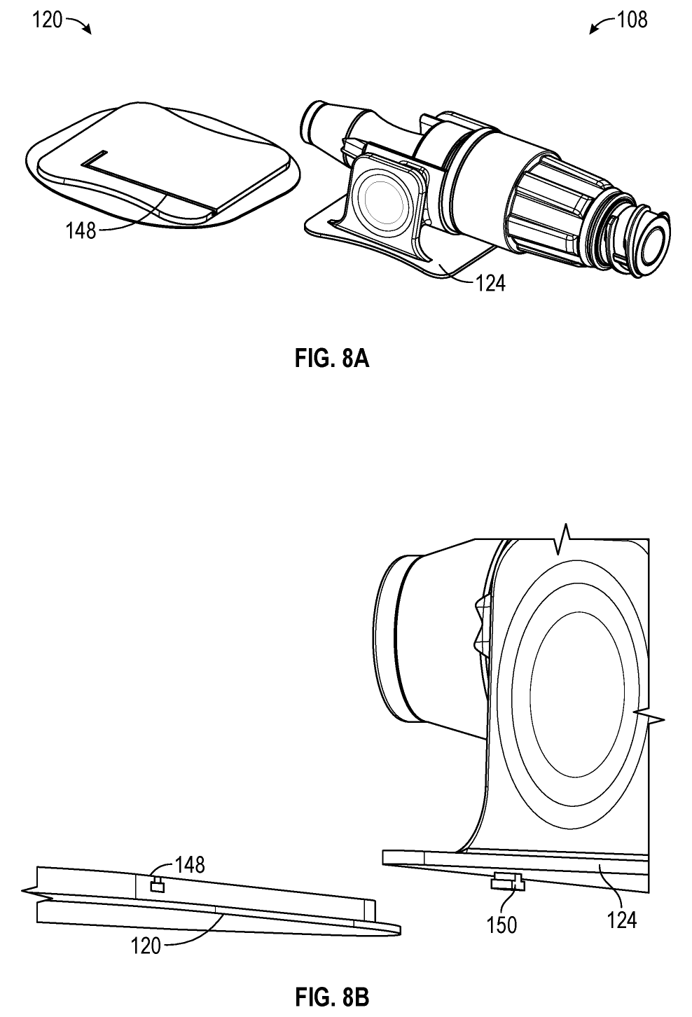

FIGS. 8A and 8B illustrate perspective views of the securement device with a key and the adhesive pad with a slot, in accordance with some aspects of the present disclosure. The slot 148 on the engagement side of the adhesive pad 120 is designed to receive a key 150, which is on the second side of the securement device 124. The coupling between the slot 148 and the key 150 holds the securement device 124 in place relative to the adhesive pad 120. In some embodiments, the securement device 124 has a slot and the adhesive pad 120 has a key.

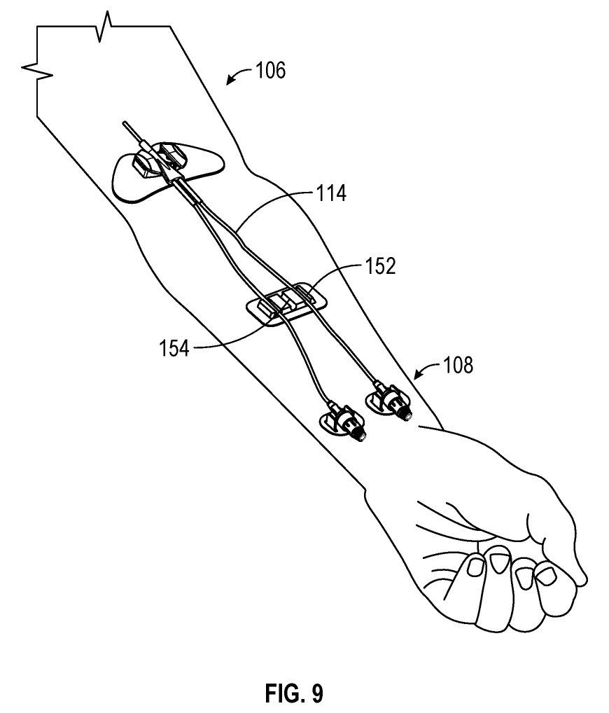

FIG. 9 illustrates an embodiment of the fluid delivery securement system that includes an extension line aligner, in accordance with some aspects of the present disclosure. The extension line aligner 152 (also referred to as the aligner 152) limits the movement of the extension lines 114 that connect the catheter hub 122 and the catheter 102. Securing the placement of the extension lines 114 is especially meaningful when the extension lines are long (e.g., extending down the length of the forearm of a patient 10). Loose extension lines 114 are likely to get tangled or caught on other medical equipment, clothing, bedding, etc. or even dislodged. When the loose extension lines 114 are tangled or caught, they are likely to get pulled or tugged on (e.g., as the patient 10 moves), which can cause abrasions to the skin of the patient 10 and introduce pathogens along the extraluminal surface through the skin insertion site. The aligner 152 secures the extension lines 114 to the arm of the patient 10 in order to limit these complications.

The aligner 152 may include an adhesive surface configured to adhere the aligner 152 to the arm of the patient 10. The aligner 152 may also include one or more channels, grooves, or slots 154 that are as approximately as wide as the width of the extension lines 114. The extension lines can be placed, snapped, or otherwise secured in the channels 144. The aligner 152 may be placed on the arm of the patient 10 between the catheter securement device 110 and the needleless connector securement system 108.

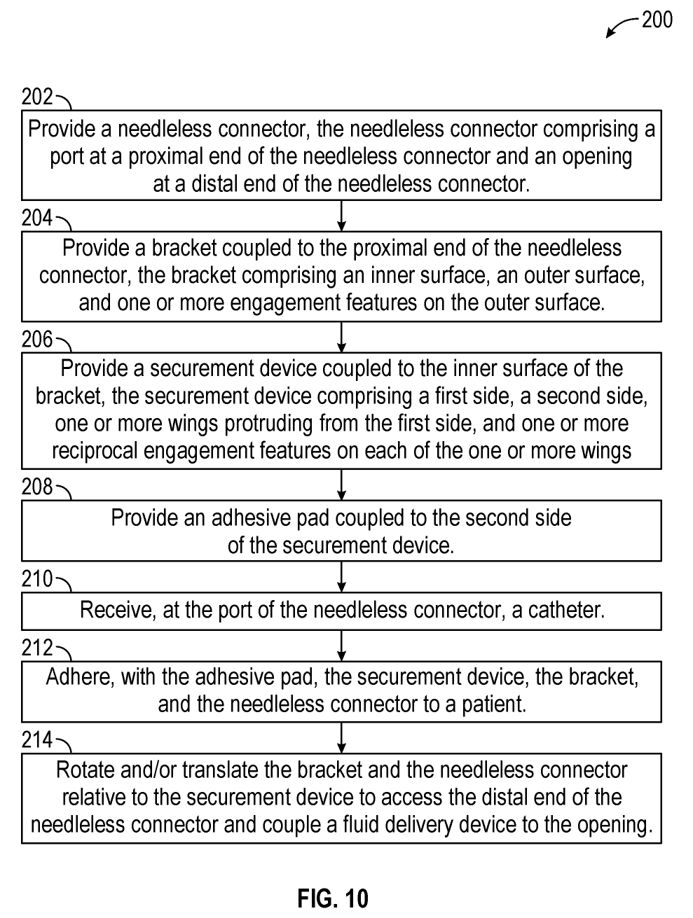

FIG. 10 illustrates a flowchart showing a method for regulating fluid delivery, in accordance with some aspects of the present disclosure. The method shown in the flowchart 200 may be performed by the fluid delivery securement systems and needleless connector securement systems described herein.

In step 202, a needleless connector is provided. The needleless connector comprises a port at a proximal end of the needleless connector and an opening at a distal end of the needleless connector.

In step 204, a bracket is provided. The bracket is coupled to the proximal end of the needleless connector and comprises an inner surface, an outer surface, and one or more engagement features on the outer surface. The needleless connector is snapped, slid, screwed, or otherwise secured to the inner surface of the bracket. The needleless connector may be interference fitted (i.e., press fitted or friction fitted) to the bracket.

In step 206, a securement device is provided. The securement device is coupled to the inner surface of the bracket and comprises a first side, a second side, one or more wings protruding from the first side, and one or more reciprocal engagement features on each of the one or more wings. The bracket and the securement device are movably coupled to each other. The engagement features and the reciprocal engagement features are coupled such that the bracket can rotate and/or translate relative to the securement device. The coupling between the engagement features and the reciprocal engagement features facilitates movement of the opening at the distal end of the needleless connector such that the opening can be moved away from the patient’s arm for ease of access.

In step 208, an adhesive pad is provided. The adhesive pad is coupled to the second side of the securement device. The adhesive pad may have an engagement side that is coupled to the second side of the securement device and an adhesive side that adheres to the patient skin in order to secure the needleless connector securement system to the patient. In some embodiments, the adhesive pad can be peeled off the second side of the securement device and replaced. In other embodiments, the adhesive pad is a reusable pad or a reusable silicone pad, which facilitates the changing of the position of the needleless connector securement system relative to the patient’s arm. In an alternative embodiment, the second side of the securement device comprises an adhesive material.

In step 210, a catheter is received at the port of the needleless connector. The catheter is removably coupled to the needleless connector.

In step 212, the securement device, the bracket, and the needleless connector are adhered to the patient by the adhesive pad.

In step 214, the bracket and the needleless connector are rotated and/or translated relative to the securement device so that the user (e.g., a patient conducting self-care in a homecare setting or a clinician) can access the distal end of the needleless connector to disinfect the needleless connector or couple a fluid delivery device to the opening at the distal end of the needleless connector. In some embodiments, one or more engagement features and one or more reciprocal features are coupled together to form a pivot, which facilitates rotation of the bracket and the needleless connector relative to the securement device and the patient’s arm. In some embodiments, one or more engagement features and one or more reciprocal features are coupled together to form a key that travels along a slot, which facilitates translation of the bracket and the needleless connector relative to the securement device and the patient’s arm. In some embodiments, one or more engagement features and one or more reciprocal features are coupled together to form a key that travels along a curved slot, which facilitates both rotation and translation of the bracket and the needleless connector relative to the securement device and the patient’s arm.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A fluid delivery securement system, the system comprising: a needleless connector comprising a port at a proximal end of the needleless connector; a bracket comprising an inner surface configured to couple to the proximal end of the needleless connector, an outer surface, and one or more engagement features on the outer surface; a securement device comprising a first side, a second side, and one or more wings protruding from the first side, wherein the one or more wings comprise one or more reciprocal engagement features for receiving the one or more engagement features on the bracket, wherein the one or more engagement features are movably coupled to the one or more reciprocal engagement features such that the bracket and the needleless connector can move relative to the securement device; an adhesive pad engageable with the second side of the securement device, wherein the adhesive pad comprises an engagement side configured to engage the adhesive pad with the second side of the securement device and an adhesive side configured to adhere the securement device to a patient; a catheter securement device comprising a different adhesive pad configured to adhere the catheter securement device to the patient; and a catheter hub coupled to the catheter securement device and comprising one or more catheters, wherein one catheter of the one or more catheters is configured to be removably coupled to the needleless connector at the port.

Clause 2. The fluid delivery securement system of claim 1, wherein the one or more engagement features and the one or more reciprocal engagement features comprise a pivot.

Clause 3. The fluid delivery securement system of claim 1, wherein the one or more engagement features and the one or more reciprocal engagement features comprise a key and a curved slot, wherein the key travels along the slot, wherein the curved slot comprises a plurality of notches configured to provide a tactile feedback when the bracket and the needleless connector are rotated and translated.

Clause 4. The fluid delivery securement system of claim 1, wherein the bracket can rotate relative to the securement device.

Clause 5. The fluid delivery securement system of claim 1, wherein the bracket can translate relative to the securement device.

Clause 6. The fluid delivery securement system of claim 1, further comprising an aligner comprising one or more channels configured to receive one or more extension lines, wherein the aligner is configured to adhere to the patient between the securement device and the catheter securement device, and wherein each of the one or more extension lines connects the one catheter to the needleless connector.

Clause 7. The fluid delivery securement system of claim 1, wherein the securement device is attached to an extension line and is configured to stow the needleless connector when the needleless connector is not in use.

Clause 8. A needleless connector securement system, the system comprising: a bracket comprising one or more engagement features and configured to receive a needleless connector; a securement device comprising a first side, a second side, and one or more wings protruding from the first side, wherein the one or more wings comprise one or more reciprocal engagement features for receiving the one or more engagement features on the bracket, wherein the one or more engagement features are movably coupled to the one or more reciprocal engagement features such that the bracket can move relative to the securement device; and an adhesive pad engaged with the second side of the securement device.

Clause 9. The needleless connector securement system of claim 8, wherein the bracket is interference fitted around the needleless connector.

Clause 10. The needleless connector securement system of claim 8, wherein the bracket comprises one or more projecting flanges that secure the needleless connector to the bracket.

Clause 11. The needleless connector securement system of claim 8, wherein the one or more engagement features and the one or more reciprocal engagement features comprise a pivot.

Clause 12. The needleless connector securement system of claim 8, wherein the one or more engagement features of the bracket comprises protrusions and/or intrusions and the one or more reciprocal engagement features of the one or more wings of the securement device comprises intrusions and/or protrusions.

Clause 13. The needleless connector securement system of claim 8, wherein the one or more engagement features and the one or more reciprocal engagement features comprise a key and a curved slot, wherein the key travels along the slot, wherein the curved slot comprises a plurality of notches configured to provide a tactile feedback when the bracket and the needleless connector are rotated and translated.

Clause 14. The needleless connector securement system of claim 8, wherein the bracket can rotate relative to the securement device.

Clause 15. The needleless connector securement system of claim 8, wherein the bracket can translate relative to the securement device.

Clause 16. The fluid delivery securement system of claim 8, wherein the adhesive pad is reusable.

Clause 17. The fluid delivery securement system of claim 8, wherein the adhesive pad comprises a reusable silicone pad.

Clause 18. The needleless connector securement system of claim 8, wherein a perimeter of the adhesive pad comprises one or more gripping ridges configured to grip one or more edges of the securement device to engage the adhesive pad to the second side of the securement device.

Clause 19. The needleless connector securement system of claim 18, wherein each of the one or more gripping ridge protrudes vertically from the adhesive pad and comprises an overhang that rests on top of the one or more edges of the securement device.

Clause 20. The needleless connector securement system of claim 8, wherein a perimeter of the adhesive pad comprises one or more guiding ridges, wherein one or more edges of the securement device are slid into and received by the one or more guiding ridges to engage the adhesive pad to the second side of the securement device.

Clause 21. The needleless connector securement system of claim 20, wherein each of the one or more guiding ridges further comprises an elastomeric rib configured to press against the securement device to engage the adhesive pad to the second side of the securement device.

Clause 22. The needleless connector securement system of claim 8, wherein the second side of the securement device comprises a key and/or slot and the adhesive pad comprises a slot and/or key, wherein the key and/or slot of the second side of the securement device is configured to fit into the slot and/or key of the adhesive pad.

Clause 23. A method for regulating fluid delivery, the method comprising: providing a needleless connector, the needleless connector comprising a port at a proximal end of the needleless connector and an opening at a distal end of the needleless connector; providing a bracket coupled to the proximal end of the needleless connector, the bracket comprising an inner surface, an outer surface, and one or more engagement features on the outer surface; providing a securement device coupled to the inner surface of the bracket, the securement device comprising a first side, a second side, one or more wings protruding from the first side, and one or more reciprocal engagement features on each of the one or more wings; providing an adhesive pad coupled to the second side of the securement device; receiving, at the port of the needleless connector, a catheter; adhering, with the adhesive pad, the securement device, the bracket, and the needleless connector to a patient; and rotating and/or translating the bracket and the needleless connector relative to the securement device to access the distal end of the needleless connector and couple a fluid delivery device to the opening.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A fluid delivery securement system, the system comprising:
a needleless connector comprising a port at a proximal end of the needleless connector;
a bracket comprising an inner surface configured to couple to the proximal end of the needleless connector, an outer surface, and one or more engagement features on the outer surface;
a securement device comprising a first side, a second side, and one or more wings protruding from the first side, wherein the one or more wings comprise one or more reciprocal engagement features for receiving the one or more engagement features on the bracket, wherein the one or more engagement features are movably coupled to the one or more reciprocal engagement features such that the bracket and the needleless connector can move relative to the securement device;
an adhesive pad engageable with the second side of the securement device, wherein the adhesive pad comprises an engagement side configured to engage the adhesive pad with the second side of the securement device and an adhesive side configured to adhere the securement device to a patient;
a catheter securement device comprising a different adhesive pad configured to adhere the catheter securement device to the patient; and
a catheter hub coupled to the catheter securement device and comprising one or more catheters, wherein one catheter of the one or more catheters is configured to be removably coupled to the needleless connector at the port.

2. The fluid delivery securement system of claim 1, wherein the one or more engagement features and the one or more reciprocal engagement features comprise a pivot.

3. The fluid delivery securement system of claim 1, wherein the one or more engagement features and the one or more reciprocal engagement features comprise a key and a curved slot, wherein the key travels along the slot, wherein the curved slot comprises a plurality of notches configured to provide a tactile feedback when the bracket and the needleless connector are rotated and translated.

4. The fluid delivery securement system of claim 1, wherein the bracket can rotate relative to the securement device.

5. The fluid delivery securement system of claim 1, wherein the bracket can translate relative to the securement device.

6. The fluid delivery securement system of claim 1, further comprising an aligner comprising one or more channels configured to receive one or more extension lines,
wherein the aligner is configured to adhere to the patient between the securement device and the catheter securement device, and
wherein each of the one or more extension lines connects the one catheter to the needleless connector.

7. A needleless connector securement system, the system comprising:
a bracket comprising one or more engagement features and configured to receive a needleless connector;
a securement device comprising a first side, a second side, and one or more wings protruding from the first side, wherein the one or more wings comprise one or more reciprocal engagement features for receiving the one or more engagement features on the bracket, wherein the one or more engagement features are movably coupled to the one or more reciprocal engagement features such that the bracket can move relative to the securement device; and
an adhesive pad engaged with the second side of the securement device.

8. The needleless connector securement system of claim 7, wherein the bracket is interference fitted around the needleless connector.

9. The needleless connector securement system of claim 7, wherein the bracket comprises one or more projecting flanges that secure the needleless connector to the bracket.

10. The needleless connector securement system of claim 7, wherein the one or more engagement features and the one or more reciprocal engagement features comprise a pivot.

11. The needleless connector securement system of claim 7, wherein the one or more engagement features of the bracket comprises protrusions and/or intrusions and the one or more reciprocal engagement features of the one or more wings of the securement device comprises intrusions and/or protrusions.

12. The needleless connector securement system of claim 7, wherein the one or more engagement features and the one or more reciprocal engagement features comprise a key and a curved slot, wherein the key travels along the slot, wherein the curved slot comprises a plurality of notches configured to provide a tactile feedback when the bracket and the needleless connector are rotated and translated.

13. The needleless connector securement system of claim 7, wherein the bracket can rotate relative to the securement device.

14. The needleless connector securement system of claim 7, wherein the bracket can translate relative to the securement device.

15. The needleless connector securement system of claim 7, wherein a perimeter of the adhesive pad comprises one or more gripping ridges configured to grip one or more edges of the securement device to engage the adhesive pad to the second side of the securement device.

16. The needleless connector securement system of claim 15, wherein each of the one or more gripping ridge protrudes vertically from the adhesive pad and comprises an overhang that rests on top of the one or more edges of the securement device.

17. The needleless connector securement system of claim 7, wherein a perimeter of the adhesive pad comprises one or more guiding ridges, wherein one or more edges of the securement device are slid into and received by the one or more guiding ridges to engage the adhesive pad to the second side of the securement device.

18. The needleless connector securement system of claim 17, wherein each of the one or more guiding ridges further comprises an elastomeric rib configured to press against the securement device to engage the adhesive pad to the second side of the securement device.

19. The needleless connector securement system of claim 7, wherein the second side of the securement device comprises a key and/or slot and the adhesive pad comprises a slot and/or key, wherein the key and/or slot of the second side of the securement device is configured to fit into the slot and/or key of the adhesive pad.

20. A method for regulating fluid delivery, the method comprising:
providing a needleless connector, the needleless connector comprising a port at a proximal end of the needleless connector and an opening at a distal end of the needleless connector;
providing a bracket coupled to the proximal end of the needleless connector, the bracket comprising an inner surface, an outer surface, and one or more engagement features on the outer surface;
providing a securement device coupled to the inner surface of the bracket, the securement device comprising a first side, a second side, one or more wings protruding from the first side, and one or more reciprocal engagement features on each of the one or more wings;
providing an adhesive pad coupled to the second side of the securement device;
receiving, at the port of the needleless connector, a catheter;
adhering, with the adhesive pad, the securement device, the bracket, and the needleless connector to a patient; and
rotating and/or translating the bracket and the needleless connector relative to the securement device to access the distal end of the needleless connector and couple a fluid delivery device to the opening.
